(19) United States
(12) Patent Application Publication
(10) Pub. No.: US 2012/0143225 A1
(43) Pub. Date: Jun. 7, 2012

(54) ENDOSCOPIC FASCIAL CLOSURE DEVICES AND METHODS FOR USING SAME

(75) Inventors: Albert K. Chin, Palo Alto, CA (US); Lishan Aklog, Scottsdale, AZ (US); Brian deGuzman, Paradise Valley, AZ (US); Rich Andrews, Lincoln, RI (US); Michael Glennon, Norwell, MA (US)

(73) Assignee: Pavilion Medical Innovations

(21) Appl. No.: 13/177,507

(22) Filed: Jul. 6, 2011

Related U.S. Application Data
(60) Provisional application No. 61/361,773, filed on Jul. 6, 2010.
(60) Provisional application No. 61/407,323, filed on Oct. 27, 2010.
(60) Provisional application No. 61/412,815, filed on Nov. 12, 2010.
(60) Provisional application No. 61/496,817, filed on Jun. 14, 2011.

(51) Int. Cl. A61B 17/04

(52) U.S. Cl. 606/148; 606/232

(57) ABSTRACT

A system is disclosed including a first expandable anchor and a second expandable anchors. The anchors may be delivered to opposing sides of a fascial opening and expanding to a predetermined size to secure the anchor at the site of delivery. A strap, designed to pull the anchors together, may extend from the first anchor to the second anchor. Upon deployment, a tension force may be exerted on the strap in order to pull the anchors toward one another and close the fascial opening. Methods and apparatus for closing the fascial opening are also disclosed.

### RELATED APPLICATIONS

This application claims priority benefit to the following applications: U.S. Provisional Patent Application 61/361,773 (filed Jul. 6, 2010); U.S. Provisional Patent Application 61/407,323 (filed Oct. 27, 2010); U.S. Provisional Patent Application 61/412,815 (filed Nov. 12, 2010); and U.S. Provisional Patent Application 61/496,817 (filed Jun. 14, 2011)—each of which is incorporated here by reference in its entirety.

### TECHNICAL FIELD

This invention relates generally to surgical closure devices and more particularly to closure devices for repairing a hernia and/or fascial defect.

### BACKGROUND ART

A hernia typically occurs in a muscle wall of an individual where the muscles have weakened, or where a previous surgical incision was made. Weakened abdominal muscles can result in a ventral hernia, which may produce a bulge or a tear forming in the surrounding tissue of the abdominal muscles. The inner lining of the abdomen can then push through the weakened area of the abdominal wall to form a hernia sack or bulge. Where a surgical incision was previously made in the abdomen, portions of the abdominal wall that have been sutured together can separate or tear between sutures over time. This also can result in the inner lining of the abdomen pushing through the tear of the abdominal wall to form a bulge or hernia sack.

Tens of thousands of ventral hernia repairs are performed in the United States each year. The conventional surgical repair procedure, or “open” method, requires that a large incision be made in the abdomen of the patient exposing the area of the hernia. The area of the hernia can be reinforced by a surgical mesh and/or closed by sutures. Since a large incision is usually made in the abdomen, the “open” method of repair can result in increased post-operative pain, an extended hospital stay, and a restrictive diet.

Laparoscopic procedures have been developed for repairing ventral hernias. These minimally invasive procedures repair the hernia opening in the abdominal wall using small incisions in the abdomen. Laparoscopes and surgical mesh are typically used in such a procedure. In particular, a mesh may be inserted through a trocar and positioned at the surgical site in the abdomen to reinforce the abdominal wall in the area of the hernia. The laparoscopic method of repair can result in decreased post-operative pain and a shorter hospital stay. However, the laparoscopic procedure can also produce some adverse affects. For example, the positioning of the surgical mesh in the abdomen can result in the mesh irritating the intestines or other abdominal contents. In addition, the surgical mesh can move in the abdomen from its original position, exposing the hernia sight and creating the potential for the development of another ventral hernia.

Sutures can also be used to close a fascial defect. Suture closure of the fascial defect, however, is typically not performed in laparoscopic ventral hernia repair, for a variety of reasons. In particular, laparoscopic suturing may be difficult to perform since manipulation of the needle takes place in a confined space with the angle of tissue access for suture placement determined by trocar port site selection. Furthermore, substantial tension is required to bring the edges of the fascia together in large ventral hernias. It may be difficult or impossible to apply a large amount of tension to suture and tie knots in the suture using present laparoscopic instrumentation. A knot is formed external to the patient, and a knot pusher pushes the knot through the trocar port down to the suture site. The process is repeated multiple times to form serial knots. In between knots, however, the knot pusher is removed from the patient and tension against the previous knot may be substantially reduced or lost, allowing the fascial defect to reopen.

Accordingly, it would be desirable to have an effective endoscopic fascial closure device that can securely couple sides of an opening or defect, while minimizing or reducing the likelihood of tearing.

### SUMMARY OF THE INVENTION

The present invention provides, in one embodiment, a system for endoscopic fascial closure. The system includes a first opposing anchor and a second opposing expandable anchor. Each anchor, in an embodiment, may be capable of being delivered through skin at the site of an opening, and capable of expanding to secure the anchor at a site of implantation. The system also includes a tensioned connector extending from the first anchor to the second anchor which, upon deployment, may be designed to pull the opposing anchors toward one another, so as to substantially pull the sides of the skin toward one another to close the opening. The device may further include a securing mechanism to secure the opposing sides of an opening in a substantially closed position. The device may further include a deployment mechanism for delivering the anchors and tensioned connector to a site of the opening. In other embodiments, the present invention includes systems, apparatuses, and methods for endoscopic fascial closure.

### BRIEF DESCRIPTION OF DRAWINGS

FIG. 1 shows a fascial closure system in accordance with an embodiment of the present disclosure.

FIG. 2 shows an anchor for fascial closure in accordance with an embodiment of the present disclosure.

FIG. 3 shows an anchor for fascial closure in accordance with an embodiment of the present disclosure.

FIGS. 4a-4b show an anchor for fascial closure in accordance with an embodiment of the present invention.

FIGS. 5a-5b show anchors for fascial closure in accordance with an embodiment of the present invention.

FIGS. 6a-6b show a fascial closure system in accordance with an embodiment of the present invention.

FIGS. 7a-7b show a fascial closure system with ratchet strap and lock in accordance with an embodiment of the present invention.

FIGS. 8a-8e show a fascial closure system including anchors having elongated members in accordance with an embodiment of the present invention.

FIG. 9 shows a delivery system in accordance with an embodiment of the present invention.

FIG. 10 shows a delivery system in accordance with an embodiment of the present invention.

FIG. 11 shows a delivery system in accordance with an embodiment of the present invention.

FIG. 12a-12b show a delivery system in accordance with an embodiment of the present invention.

FIG. 13 shows an ejection mechanism in accordance with an embodiment of the present invention.

FIG. 14 shows a fascial closure system and tensioning system in accordance with an embodiment of the present invention.

FIGS. 15a-h show embodiments of a tensioning system for fascial closure.

FIGS. 16a-16b show a tensioning system in accordance with an embodiment of the present invention.

FIGS. 17a-17b show a tensioning system in accordance with an embodiment of the present invention.

FIGS. 18a-18c show a crimping mechanism in accordance with an embodiment of the present invention.

FIGS. 19a-19c show a cutting mechanism in accordance with an embodiment of the present invention.

FIGS. 20a-20f show a pulling and cutting mechanism in accordance with an embodiment of the present invention.

### DESCRIPTION OF SPECIFIC EMBODIMENTS

In accordance with one embodiment of the present invention, systems and methods are provided herein for closure devices for use in connection with fascial closures. A closure system 100 for holding a fascial defect closed may be provided. The closure system 100 may be delivered into the body of a patient through one or more laparoscopic incisions by a delivery system 900. Once in place, a tensioning mechanism 1400 may be used to create and maintain tension on the closure system 100, thus pulling the fascial defect closed and holding it in a closed position.

The endoscopic and/or laparoscopic fascial closure devices of the present invention may find use in, for instance, repairing a ventral hernia. Although discussed herewith in connection with a ventral hernia (i.e., opening in the abdominal wall), it should be appreciated that the device of the present invention can be adapted for use to close other openings in the body. For example, the endoscopic fascial closure device of the present invention may also find use in other types of hernias, or in other types of openings in tissue or organs.

Closure System

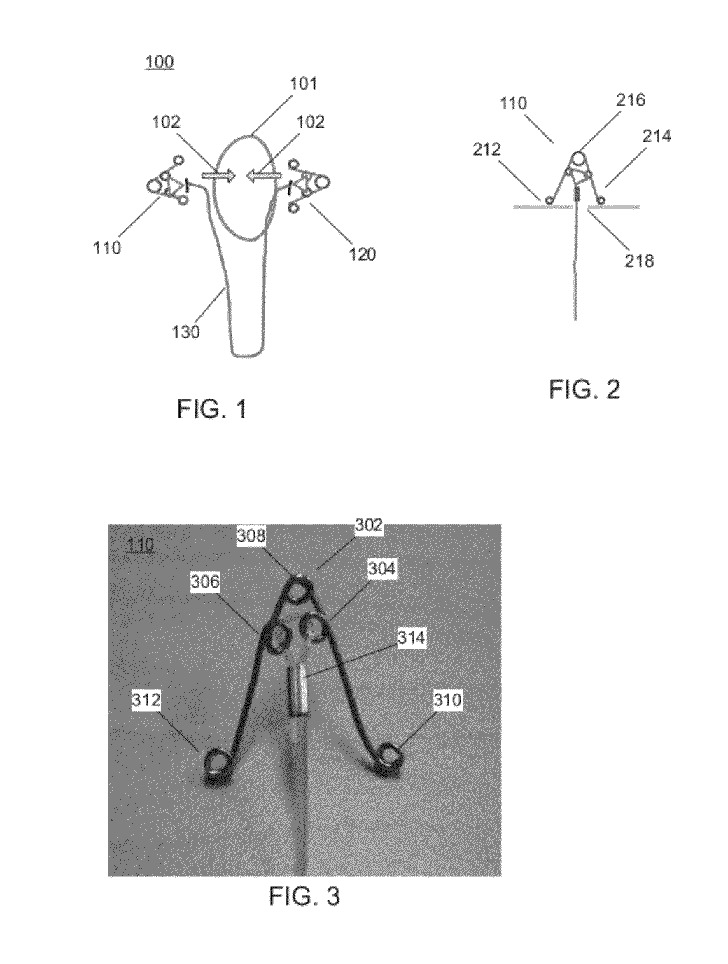

FIG. 1 illustrates a closure system 100, in accordance with one embodiment of the present invention, deployed at the site of a fascial opening 101. Closure system 100 may be designed to exert and maintain a closing force on or around opening 101 so that opening 101 can be closed in the manner shown by arrows 102. In an embodiment, closure system 100 may include a first anchor 110 and a second anchor 120, designed to be positioned on opposing sides of opening 101. Closure system 100 may also include a tensioning mechanism, such as a connector or strap 130, coupled at each of its ends to one of the anchors. In that way, once anchors 110 and 120 are in position, a force may be applied to strap 130 in order to pull anchors 110 and 120 toward one another, in the direction shown by arrows 102. As anchors 110 and 120 are drawn together, they act to pull the opposing sides of opening 101 toward one another to close opening 101. In one embodiment, anchors 110 and 120 may be pulled together by placing strap 130 under tension. As discussed below in greater detail, strap 130 may be a suture, strap, elongate member, or any other device capable of pulling anchors 110 and 120 toward each other in order to close opening 101.

Anchors

Closure system 100 may include one or more anchors designed to be fasten near the site of a fascial defect. In accordance with one embodiment of the present invention, first anchor 110 and second anchor 120 may be collapsible so that, in a collapsed mode, the anchors can have a slim or thin profile in order to facilitate their deployment into a patient, for example, through a small incision. Anchors 110 and 120 may also be expandable so that, once deployed through a small incision, anchors 110 and 120 may expand to maximize their anchoring properties around a fascial defect. For example, anchors 110 and 120 may be designed to have a V-shape as illustrated in FIG. 2. Anchor 110, for instance, may include two proximal ends 212 and 214 and an apex 216. Apex 216, in an embodiment, may act as a pivoting point to allow the proximal ends 212, 214 to expand away from each other and/or collapse toward each other. When anchor 110 is in a collapsed mode, proximal ends 212, 214 may be adjacent to each other so that anchor 110 has a substantially thin profile and can more easily fit through a small incision in the skin, such as incision 218.

In another embodiment, anchors 110 and 120 may have fixed barbs or hooks at a transverse angle so that, once placed at a site if implantation, they can hook into the surrounding tissue so they do not move.

Once anchor 110 is placed through the incision, anchor 110 may expand so that proximal ends 212 and 214 can move away from one another to permit the ends to be spatially situated relative to one another, thereby providing anchor 110 with a substantially wider profile than that of incision 218. In this way, anchor 110 may be securely deployed at an area near the fascial opening. Although reference is made to anchor 110, it should be appreciated that similar features and designs are provided in connection with anchor 120 or other anchors, so as to permit anchor 120 or other anchors to perform in a similar manner.

Anchors 110 and 120, in one embodiment, can be provided with any size, depending on the application and size of the incision 218. It should be noted that the width or profile of the anchors 110 and 120 should also permit the anchors 110 and 120 to fit within any suitable trocar, catheter, needle or other delivery mechanism for insertion into the body. In one embodiment, the anchors 110 and 120 may have a collapsed width or profile sufficient to load the anchors 110 and 120 into the lumen of a tapered 3 mm diameter needle.

In an embodiment, anchors 110 and 120 may be formed from a continuous piece of wire arranged in a V-shape as shown, for example, in FIG. 3. In the V-shape, the wire can form at least two legs extending from apex 302. It should be noted that these legs can be of varying length. Of course, other designs may also be possible. It should also be appreciated that while described here as forming a V-shape and having two legs, other designs with a greater number of legs may also be possible as the present invention is not intended to be limited in this manner.

As shown in FIG. 3, attachment points, such as eyelets 304 and 306, may be provided along the length of the anchor and/or at apex 302. These attachment points may also act as a pivot point or may operate to increase anchoring properties. Eyelets 304 and 306, in an embodiment, may be designed to provide an attachment area for a suture (described in detail below). In one embodiment, one eyelet may be provided on each leg, such as eyelets 304 and 306, while one eyelet may be provided at the apex, such as eyelet 308. These eyelets, as noted, may have various functions. For instance, eyelet 308 at apex 302 of anchor 110 may act as a pivoting point and may provide anchor 110 with the ability of flex, that is collapse or expand. Eyelets 304 and 306, on the other hand, may be situated in such a location that tensioning or pulling against eyelets 304 and/or 306 may occur without collapsing the anchor 110. Eyelets 310 and 312 at the proximal ends of the legs may provide a smooth surface to prevent or minimize the likelihood of the legs penetrating the skin, as well as providing a relatively large surface to maximize anchoring to or beneath the skin. One skilled in the art will recognize that strap 130 or other connectors may be attached to any or all of the eyelets in an anchor. Also, although reference is made to anchor 110, one skilled in the art will recognize that the structures, features, and functions described may also apply to anchor 120 and/or other anchors.

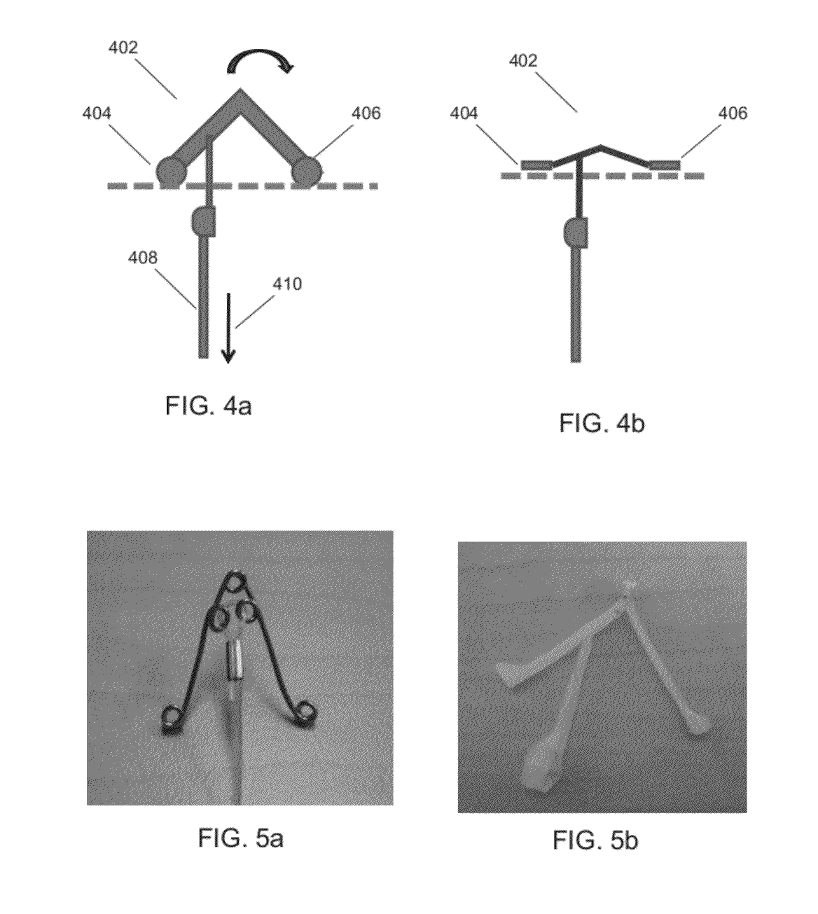

In some embodiments, anchors 110 and 120 may expand to a substantially flat position in order to reduce the possibility that the anchors will inadvertently become removed from the body through the incision and secure the anchor in its position near the fascial defect. For example, the legs of anchors 110 and 120 may expand to form an obtuse angle and/or a 180 degree angle. Ss shown in FIGS. 4a and 4b, with the proximal ends 404 and 406 spaced apart from one another, anchor 402 may be securely placed in its location. In this expanded position, the proximal ends 404, 406 of anchor 402 can be spaced apart relatively wider than the width of the incision, thus allowing the anchor 402 to secure itself against the skin or fascia, thereby avoiding or reducing any potential for being pulled out. The proximal ends 404 and 406, along with apex 402, in one embodiment, can be designed to allow anchor 404 to spread and lie flat against the fascia, as shown in FIGS. 4a and 4b, to maximize its anchoring properties. Such a design can allow for tensioning of the closure system 100 to occur inside the body, for instance, the abdomen. In an embodiment, exerting a tensioning force on strap 408, as shown by arrow 410, can cause anchor 402 to spread open to move proximal ends 404 and 406 away from each other, until anchor 402 lies substantially flat against the tissue.

In some embodiments, to adequately secure the anchors 110 and 120 to the sides of the opening, the anchors of the present invention may be made from a material and provided with a design that can bias to an open or expanded position. In another embodiment, the anchors 110 and 120 may be formed from a self-expanding, shape memory, biasing or spring material. Examples of such self-expanding, shape memory, biasing or spring material include, but are not limited to, metal, metal alloy, polymer, molded plastic, metal-polymer blend, or a combination thereof. As the type of material used can also affect the strength and/or flexibility of the anchors 110 and 120, examples of suitable materials include stainless steel, gold, platinum, tungsten, nickel-titanium alloy, Beta III Titanium, cobalt-chrome alloy, cobalt-chromium-nickel-molybdenum-iron alloy, a substantially rigid plastic material such as Ultem, nylon or polycarbonate, or any other suitable material that is biocompatible and that is capable of being expanded in the manner described above. The system 100 may also include an anti-thrombogenic coating such as heparin (or its derivatives), urokinase, or PPack (dextrophenylalanine proline arginine chloromethylketone) to prevent thrombosis or any other adverse reaction from occurring at the site of insertion. FIGS. 5a and 5b show examples of anchors formed from different materials. In particular, FIG. 5a illustrates an anchor made from metal, while FIG. 5b illustrates an anchor made from a hard plastic material.

Since the closure system 100 for endoscopic fascial closure can be designed to be implanted within a human or animal body, the system 100 may be made from a material that is biocompatible. The biocompatibility of the material may help minimize occurrence of adverse reactions due to implantation of the system 100 within the body. In some embodiments, the system 100 can be made entirely or partially from material that is bioresorbable, or biodegradable, or a combination thereof. In such instances, the system 100 may be entirely or partially absorbed by the vessel or may be degraded after a certain period of time has elapsed, and would eliminate the need for manual removal of the system 100.

In one embodiment, the anchors 110 and 120 can be molded or formed as a one-piece design. In such a design, the anchors 110 and 120 can be molded out of a plastic material such as nylon, polycarbonate, Ultem, or other types of plastic materials. Molding the anchors 110 and 120 as one piece design can act to maximize the tensile strength of each unit, leading to enhanced closure of the fascial defect.

Tensioning Strap and Lock

In accordance with an embodiment of the present invention, a connector or strap, such as strap 130 shown in FIG. 1, may be provided between anchors 110 and 120. Strap 130 may be connected to anchors 110 and 120, and may be designed to be placed under tension in order to pull anchors 110 and 120 toward one another once they are in position on opposite sides of opening 101, As illustrated in FIG. 1, the presence of strap 130 between the anchors allows strap to pull or otherwise move anchors 110 and 120 toward each other in order to pull the sides of opening 101 together to partially or completely close the fascial defect.

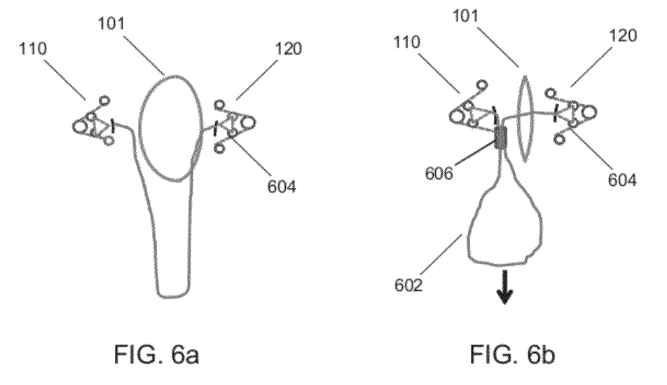

In an embodiment, looking now at FIGS. 6a-6b, strap 130 may be a suture 602. The suture 602, as shown in FIGS. 6a and 6b, may extend from the first anchor 110 to the second anchor 120, and may be connected to anchors 110 and 120. In one embodiment, the suture 602 can be directed through eyelets 604 and/or other eyelets provided, for example, at a mid-portion of anchors 110 and 120. Alternatively, suture 130 may be attached to the anchor 110 in the manner shown in FIG. 3. In this instance, the suture 130 may be threaded through two eyelets 304, 306 in the mid-portion of the anchor 110 and may be secured, for instance, by a crimped tube 314. By being threaded through the eyelets in the mid-portion of the anchor 110, as, tensioning or pulling of the suture 602 may occur without collapsing the anchor 110 and without allowing suture 602 to become free of the anchor 110. If the suture 602 were to be connected only to the apex 302 of the anchor 110, for instance, tension on the suture may cause the apex 302 to be directed to the entry site in the fascia, and may result in the anchor 110 being pulled through the fascia.

To adequately maintain the opening substantially closed, the suture 602 may be made from a material that is relatively strong, so that it does not break when placed under tension sufficient to close opening 101. Additionally, the suture 602 may be made from a material that is biocompatible. The biocompatibility of the material may help minimize occurrence of adverse reactions due to implantation of the system 100 within the body.

In one embodiment, the suture 602 can also be made from a material that allows for its subsequent elimination once the fascial closure function is no longer necessary. As used herein, the term “elimination” can be understood to mean manual removal of the element or otherwise. In one embodiment, the suture 602 can be made from a material that is capable of being severed. Such a material would allow for manual removal of the suture 602. In other embodiments, the suture 602 can be made entirely or partially from material that is bioresorbable or biodegradable. In such instances, the suture 602 may be entirely or partially absorbed by the body after a certain period of time had elapsed and would eliminate the need for manual removal of the suture 602. Examples of suitable materials include catgut suture, silk, polyglycolic acid, polylactic acid, polydioxanone, nylon, polypropylene, or a combination thereof.

In accordance with another embodiment, the closure system 100 may further include a securing mechanism 606 as illustrated in FIG. 6b, to maintain the opening 101 in a substantially closed position. In an embodiment, securing mechanism 606 may be a sleeve for crimping around strap 130 in order to hold strap 130 under tension. For example, once strap 130 is under tension and anchors 110 and 120 have been pulled together, securing mechanism 606 may be used to maintain the tension on strap 130 so that the now-closed opening 101 does not revert back to its open position.

In an embodiment, as shown in FIG. 6b, the securing mechanism 606 may be a hollow object, such as a tube, that is designed to be advanced forward along the suture 602 toward anchors 110 and 120. Once situated at a position adjacent to the anchors 110 and 120 such that the opening 101 is maintained in a substantially closed position, the securing mechanism 606 may be crimped in such a manner so as to prevent movement of the securing mechanism 606 while maintaining the opening 101 in a substantially closed position. In one embodiment, once crimped around suture 602, securing mechanism 606 may prevent suture 602 from slipping through securing mechanism 606, thus maintaining necessary tension between anchors 110 and 120. While described as a tube, the securing mechanism 606, of course, may have any other geometric shape as well. It should further be noted that the securing mechanism 606 may be made from any substantially hard or strong material to allow the securing mechanism 606 to maintain its position and prevent the mechanism 160 from breaking, stretching, or otherwise weakening.

In an embodiment, strap 130 may have a roughened surface so that it does not slip under tension. For example, strap 130 may be a weaved or braided suture where the weaves and/or braids generate friction to prevent slippage. In another embodiment, the surface of the strap may include beads, bumps, teeth, or other protrusions, or a combination thereof, against which the locking mechanism can engage in order to prevent or reduce slippage of the strap.

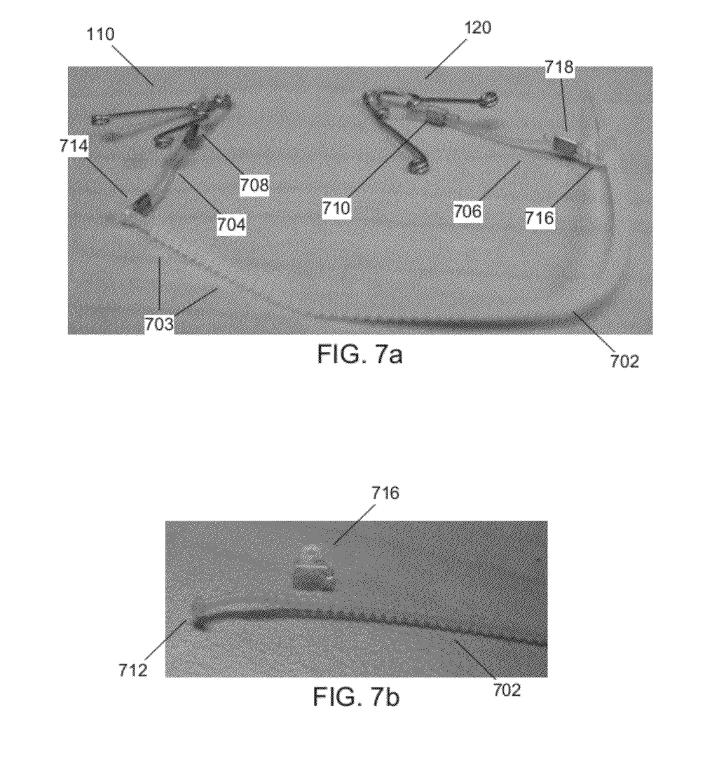

In another embodiment, the securing mechanism may be a ratchet lock, and the strap 130 between anchors 110 and 120 may be designed to act with the ratchet. to create and maintain tension between anchors 110 and 120 by means of a ratcheting action. FIGS. 7a and 7b show a strap 702 with such a ratchet design. In an embodiment, ratchet strap 702 may have bumps or teeth 703 along its surface to facilitate ratcheting and securing of ratchet strap 702 between anchors 110 and 120. The teeth 703, which can be uni-directional, may provide traction, friction and/or ratcheting action, so that ratchet strap 702 can pull and hold anchor 110 and anchor 120 in position to facilitate closure of opening 194.

When utilizing ratchet strap 702, anchor 110 and anchor 120 may include, in an embodiment, connectors 704 and 706 respectively, to secure anchors 110 and 120 respectively to ratchet strap 702. As shown, connector 704 may be secured to anchor 110 by coupling one of its ends, for instance, to those eyelets in the mid-portion of anchor 110, and may employ bushing 708 to maintain that particular end through the eyelets. In particular, bushing 708 may crimp or otherwise secure the end of connector 704 so that, once looped through the eyelets of anchor 110, connector 704 does not become free from anchor 110. Connector 706 may be secured to anchor 120 in like fashion by utilizing bushing 710. Although illustrated and described as a connector, connectors 704 and 706 may include any mechanism or design capable of coupling anchors 110 and 120 to ratchet strap 702.

The opposing end of connector 704 (i.e. the end that is distal to anchor 110) may be looped about a retaining mechanism, such as button 712, on ratchet strap 702. Button 712, as illustrated, may be provided with a design such that when the opposing end of connector 704 is looped thereabout, the ratchet strap 702 can be prevented from slipping through such a loop. As illustrated in FIG. 7a, such a loop at the opposing end of connector 704 can be maintained by bushing 714. In that manner, when ratchet strap 702 is under tension, retaining button 712 may act as a stop knot, so that ratchet strap 702 cannot be pulled from the loop in connector 704. In an embodiment, button 712 may be a protrusion at the end of ratchet strap 702 and may have a diameter larger than ratchet strap 702 and/or larger than the diameter of the loop in connector 704.

The opposing end of connector 706 (i.e. the end that is distal to anchor 120) may be attached to a locking mechanism. The locking mechanism may engage strap 130 so as to maintain tension on the strap and reduce the chance that the strap will become slack. In an embodiment, the locking mechanism may be movable along the length of the strap so as to adjust the tension on the track. In some embodiments, the locking mechanism may be a ratcheting device, such as ratchet lock 716 through which ratchet strap 702 can be advanced. As shown in FIG. 7a, connector 706 may be coupled to ratchet lock 716 and held fast by bushing 718. In other embodiments, locking mechanism may be a sleeve that can be crimped about strap 130. In yet another embodiment, the locking mechanism may be a cinch that can tightly cinch or bundle the strap together so that Ratchet lock 716, in one embodiment, may include a mechanism which, when interacting with the bumps or teeth 703 on the ratchet strap 702, allows ratchet strap 702 to be pulled through in one direction in order to increase tension on ratchet strap 702, but not allow ratchet strap 702 to move in the other direction to release tension. Additionally or alternatively, ratchet lock 716 may have a locking mechanism so that once locked, ratchet strap 702 is held fast by ratchet lock 716. In this way, ratchet strap 702 and ratchet lock connector 716 may create and hold tension between anchors 110 and 120, in order to facilitate closure of opening 101. In some embodiments, ratchet strap 703 may have uni-directional teeth 703 (teeth with a saw-tooth design, for example), so that ratchet strap 702 can advance through ratchet lock 716 in a single direction in order to increase tension, but is prevented from withdrawing from ratchet lock 716 in the opposite direction.

It should be appreciated that although both connectors 714 and 716 have been described in connection with the use of a bushing to maintain the connectors coupled to the respective anchors at one end, and to the ratchet strap or ratchet lock at the opposing end, any other mechanisms or designs known in the art can be used, so long at the connector can be secured in the manner intended.

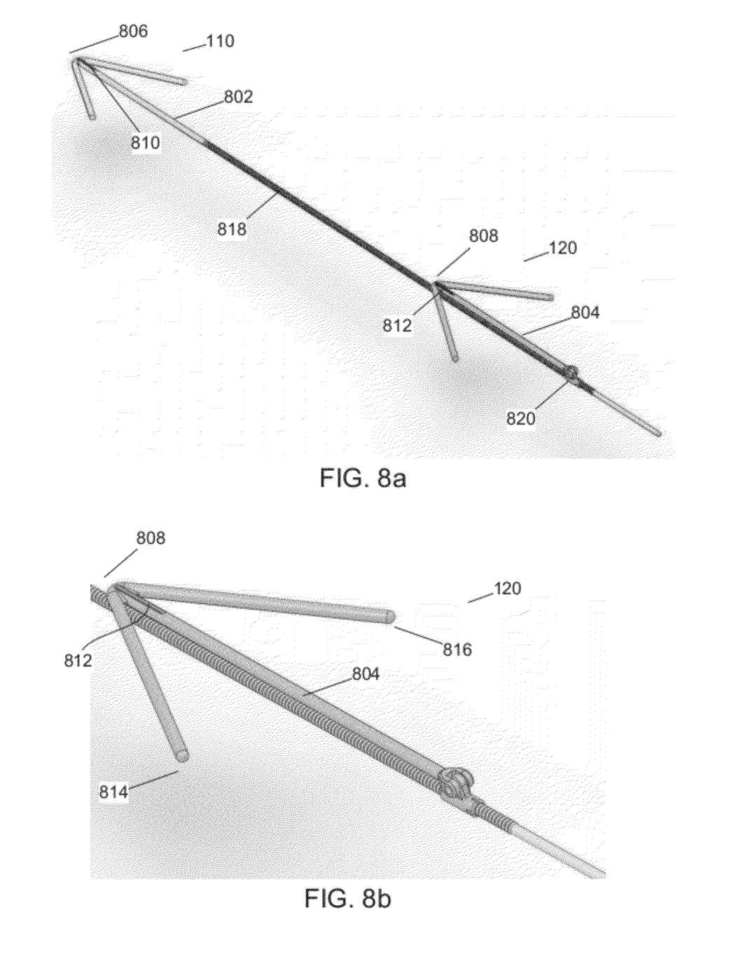

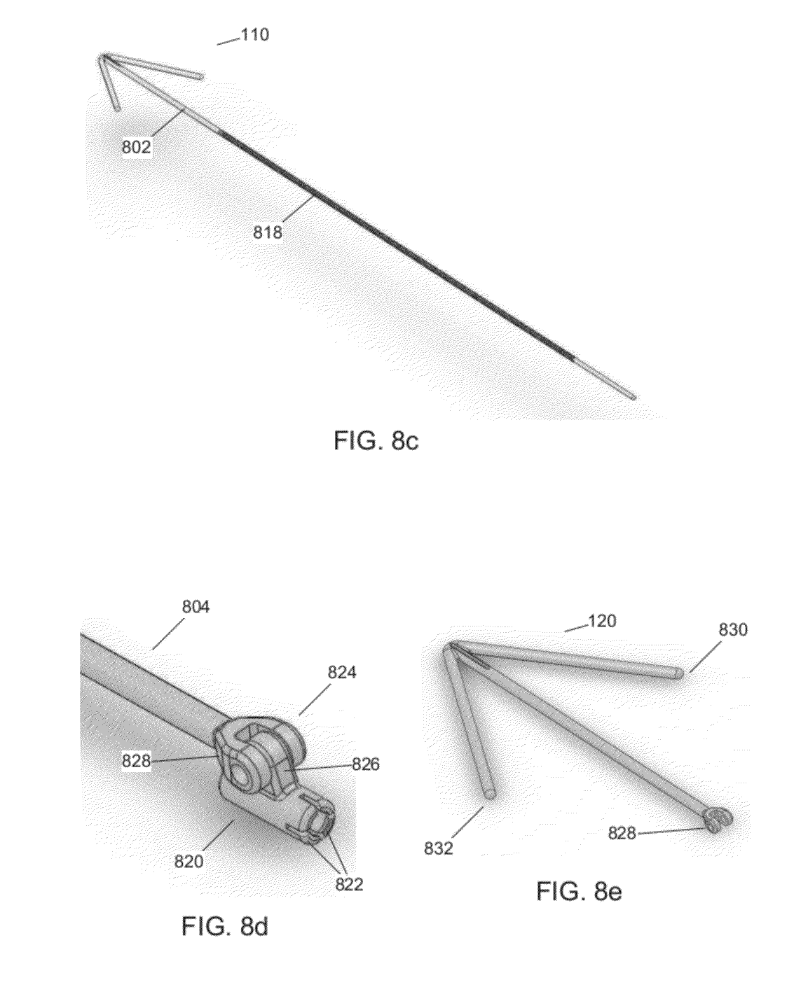

With reference now to FIGS. 8a-8e, there is shown another embodiment of the closure system 100 of the present invention. As shown therein, first anchor 110 and second anchor 120 may include respective elongated member 802 and 804 designed to permit anchors 110 and 120 to be pulled toward one another. Elongated members 802 and 804, in an embodiment, may be connected to anchors 110 and 120 respectively at apex 806 and 808. In some instances, each of the elongated members 802 and 804 can include, at the apex-connecting end, a stiffening geometry 810, 812, respectively, to provide the anchor with added strength. For example, the stiffening geometry 812, as illustrated in FIG. 8b, can increase the connecting strength between the apex 808 and the elongated member 804, so as to increase the tension at the apex 808 and/or the stiffness of the anchor 120. The resulting connecting strength, tension, and/or stiffness can provide added strength that can allow the proximal ends 814 and 816 to sufficiently spread without completely collapsing. This can also minimize any pulling or slipping of the anchors away from the site of placement. The stiffening geometry, in an embodiment, can be formed from any sufficiently strong material generally known in the art, including, but not limited to, metal, metal alloy, polymer, molded plastic, metal-polymer blend, or any combination thereof. In an embodiment, the stiffening geometry can be a metal insert. The stiffening geometry can also be molded out of a plastic material such as nylon, polycarbonate, Ultem, or other types of plastic materials.

In accordance with an embodiment of the present invention, the first anchor 110 and the second anchor 120 can be provided with complimentary mechanisms to permit advancement of the opposing anchors 110 and 120 toward one another. In that way, the opposing anchors 110 and 120 can act to pull the sides of the fascia toward one another to substantially close an opening therebetween. In one embodiment, the anchors 110 and 120 can include a strap and lock mechanism, similar to those disclosed above. To that end, strap 818 and lock 820 can extend from the elongated members 802, 804, respectively. For example, strap 818 and lock 820 can extend from the elongated members 802 and 804, respectively, adjacent to the end opposing the apex-connecting end thereof. As illustrated in FIG. 8a, the elongated member 802 can be of an extended length where a portion of the elongated member 802 can be used as strap 818 that can be complimentarily received by lock 820. The strap 818, in an embodiment, can be provided with teeth to facilitate pulling and securing of the anchors 110 and 120.

As shown in FIG. 8b, the strap 818 can include teeth (e.g., uni-directional teeth), and can be pulled through the lock 820 (e.g., a ratchet lock, a snap-fit lock) to bring the sides of the fascia together. Upon tensioning of the strap 818, the closure system 100 can be designed to allow the anchors 110 and 120 to move toward one another, while allowing each of their respective opposing proximal ends to sufficiently spread against the fascia to maximize their anchoring properties. In some embodiments, strap 818 and lock 820 may include a ratchet mechanism, such as the ratchet mechanism described above. In such an embodiment, strap 818 may include bumps or teeth for increasing friction and/or providing a ratcheting action. Similarly, lock 820 may include a ratchet mechanism so that, as strap 818 is advanced through lock 820, lock 820 allows the strap to advance, but prevents the strap 818 from reverse motion and pulling out of lock 820. In some embodiments, the bumps or teeth may be uni-directional (e.g. a saw-tooth design) to allow movement through lock 820 and prevent strap 818 from pulling out of lock 820. It should be appreciated that while being described as having a strap and lock and/or ratchet mechanism, the present invention can be provided with other complimentary mechanisms as well. For instance, the anchors 110 and 120 can include a modified cable tie mechanism.

The strap 818 and elongated member 802, in an embodiment, can be formed as a one-piece unit (e.g., molded together) or as two detachable parts. The lock 820 and elongated member 804, in an embodiment, can also be formed as a one-piece unit (e.g., molded together) or as two detachable parts. The strap 818 and/or lock 820 can be made from the same or different materials than the elongated members 802 and 804. For example, the strap 818, lock 820, and/or elongated members 318 and 328, in an embodiment, can be formed from a flexible material that can allow shaping, molding, expanding, adapting, bending, twisting, turning, tensioning, pulling, and/or manipulating. The material can be sufficiently rigid to allow pulling of the anchors 110 and 120. In one embodiment, the strap 818, lock 820, and/or elongated members 802, 804 can be formed from a self-expanding, shape memory, biasing or spring material such as metal, metal alloy, polymer, molded plastic (e.g., nylon, polycarbonate, Ultem), metal-polymer blend, or any combination thereof.

In one embodiment, the anchor 110/strap 818 unit and anchor 120/lock 820 unit can also be molded or formed as a one-piece design. For example, anchor 110 and strap 818 can be formed or molded as a one-piece, as shown in FIG. 8c where the strap 818 can be an extension of the elongated member 802. In another example, a portion of the elongated member 802 can be provided with teeth and be used as a strap. Anchor 120 and lock 820 can also be formed or molded as one-piece. In an embodiment, the anchor 110/strap 818 unit and anchor 120/lock 820 unit can be molded out of a plastic material such as nylon, polycarbonate, Ultem, or other types of plastic materials. Molding the anchor 110/strap 818 unit as one piece, and the anchor 120/lock 820 unit as one-piece can act to maximize the tensile strength of each unit, leading to enhanced closure of the fascial defect.

The anchor 120/lock 820, in an embodiment, can be designed so that the lock 820 can complimentarily receive the strap 818. In particular, the lock 820 can be placed at the end of the strap 818 that is opposing the first anchor 110. Upon tensioning of the strap 818, the lock 820 can be designed to allow the lock 820 to move along the strap 818 toward the first anchor 110, so as to allow the anchors 110 and 120 to move toward one another. The lock 820 can be designed to move along the strap 818 uni-directionally or bi-directionally. The lock 820 can also be designed to rotate or revolve around the strap 818. In one example, the lock 820 can include two or more (e.g., four) flexible parts 822 at one end, as shown in FIG. 8d. The flexible parts 822 can be configured to form, for example, a snap-fit type of lock, which at a substantially tension free state can grip and lock in its position (e.g., in a groove between the teeth), and upon tensioning can expand outwardly so as to allow the lock 820 to move along the teeth on strap 818 (e.g., moving from one groove to the next).

In accordance with an embodiment as illustrated in FIGS. 8d and 8e, the second anchor 120 can be pivotably attached to the lock 820 via a pivot 824. The anchor 120 can pivot around the pivot 824 and change its orientation from, e.g., being opposite to the first anchor 110 to, e.g., being in substantially the same direction as the first anchor 110, or anywhere in between. One advantage of including the pivot can be that when the anchors 110 and 120 are in substantially the same direction, it is easier to fit the system into a delivery mechanism (e.g., a cannula, a trocar, a catheter, or a needle). Once the system is discharged or delivered to a site of interest, the anchor orientation can be changed by pivoting the anchor such that the anchors can be positioned at an optimal angle against the skin or fascia. In addition, an optimal anchor orientation can be selected by pivoting the anchor, depending on the position of the anchor and the size of the fascial defect. The pivot can also allow the adjustment of the anchor orientation in accordance with the advancement of the anchors 110 and 120 toward one another. In some embodiments, both of the anchors 110 and 120 can be designed to include a pivot such that the orientation of the anchor 110 and/or 120 can be changed in relation to one another.

Any suitable pivot known in the art can be used for connecting the anchor 120 and the lock 820. For example as shown in FIGS. 8d and 8e, the pivot 824 can include a pair of pivoting parts held together with a pin or bolt, with a first pivoting part 826 (e.g., a mount or rod end attached to the lock 820) positioned between two flanges of a second pivoting part 828 (e.g., a bracket), with the flanges having surfaces mating with side surfaces of the rod end. The pivoting parts 826 and 828 can have a pivot hole therethrough for insertion of the pin or bolt. A lock nut can also be include to hold the pin or bolt and the two pivoting parts together.

In some embodiments, the anchors 110 and 120 can be designed to transition from a first position, where the anchors 110 and 120 are collapsed with the proximal ends adjacent to one another, to a second position, where the anchors 110 and 120 are expanded with the proximal ends spaced apart from one another. In the first position, the anchors 110 and 120 can be designed to have a sufficiently small width or profile to allow the anchors 110 and 120 to be delivered through a slit in skin, or fascia, adjacent to an opening. The anchors 110 and 120 can be provided with any size, depending on the application and size of the opening. It should be noted that the width or profile of the anchors 110 and 120 can also permit the anchors 110 and 120 to fit within any suitable trocar, catheter, needle or other delivery mechanism for insertion into the body.

In a second position, with the proximal ends spaced apart from one another (e.g. as shown in FIG. 8e, where proximal ends 830 and 832 are shown spaced apart from each other), anchors 110 and 120 can act to secure the anchor 110 or 120 at the site of implantation. In this expanded position, the proximal ends (e.g. proximal ends 830 and 832) are expanded wider than the width of the incision in the patient, allowing the anchors 110 and 120 to secure themselves against the skin or fascia, avoiding or reducing any potential for pullout of the fastener. The proximal ends 830 and 823, for example, can be designed to allow the anchors 110 and 120 to spread and lie substantially flat against the fascia to maximize their anchoring properties. Such a design can allow for tensioning of the closure system 100 to occur inside the body, for instance, the abdomen.

The tensioning strap, e.g. the suture, ratcheting strap, elongate members, etc., may be constructed of biocompatible, bioresorbable, and/or biodegradable material so that the monofilament strands and/or the ratcheting strap may be tolerated, or eliminated from the body, and/or partially or entirely absorbed by the body once the closure force is no longer required.

Similarly, the bushings described above, in an embodiment, may be constructed of a malleable material, such as steel or another metal, so that it may be crimped in place to secure the end of the various connectors, straps, etc., for example. Alternatively, the bushings may be constructive of a biocompatible material, and/or a bioresorbable or biodegradable material, so long as the bushings can secure the ends of the monofilament strands, as described above.

Delivery System

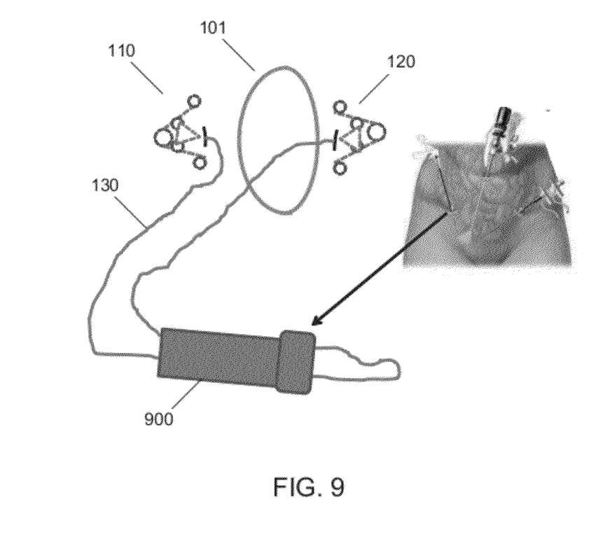

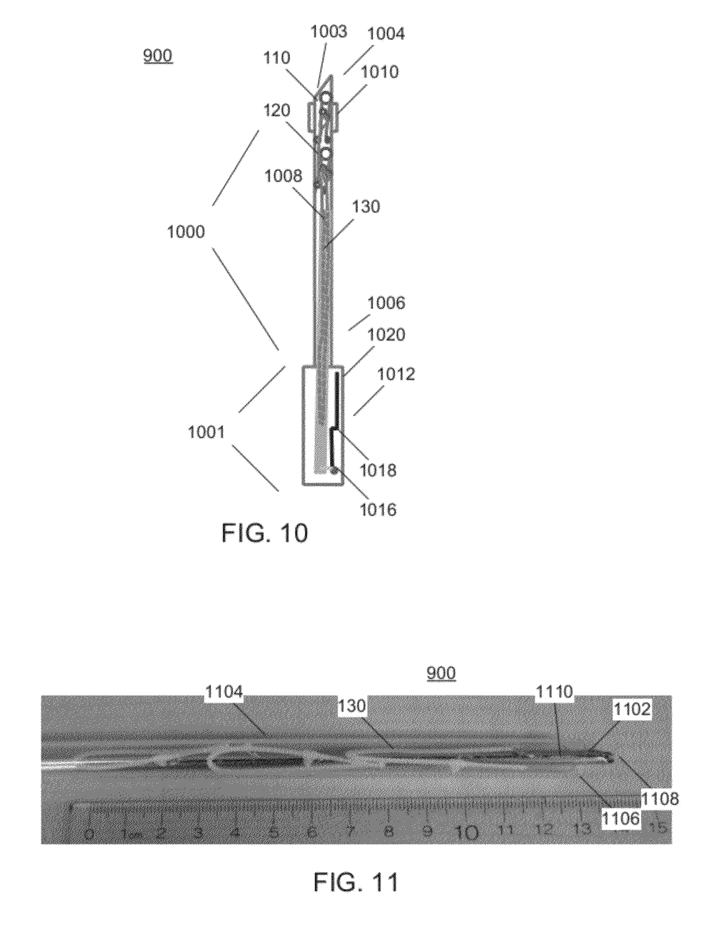

Now referring to FIGS. 9-10, a delivery system 900 is provided for delivering closure system 100 to a site of implantation. Delivery system 900, as illustrated, may include a chamber 1000 for accommodating closure system 100 prior to delivery, and an ejection mechanism 1001 designed to discharge closure system 100 from the chamber to the site of implantation. Delivery system 900, in one embodiment, may be designed to deliver closure system 100 to a site of implantation through small incisions in the patient, as shown in FIG. 9. In some embodiments, the delivery system 900 may be an endoscopic, laparoscopic, or other minimally invasive delivery system designed to deliver closure system 100 through relatively small incisions in the patient. However, one skilled in the art will recognize that system 100 and the associated delivery system 900 may also be used during laparotomy, coeliotomy, etc., or other surgeries using large incisions.

In accordance with another embodiment of the invention, the delivery system 900, as illustrated in FIG. 10, can include a chamber 1000 for accommodating closure system 100 and for delivering closure system 100 to a site of interest in a patient. In an embodiment, chamber 1000 may be defined by a tube or housing, such as tube 1002, having a delivery end 1004, an opposing end 1006, and a passageway 1008 therebetween. Tube 1002 may have an opening 1003 at delivery end 1004 to allow closure system 100 to be directed from chamber 1000 into a patient. Opening 1003, in one embodiment, may have a diameter sufficiently large to allow closure system 100 to pass therethrough, while being sufficiently small so that delivery end 1004 may be inserted through a laparoscopic incision in the patient for delivery of closure system 100 to the site of a fascial defect. In another embodiment, if no incision is present, opening 1003 may be tapered and/or sharp to allow delivery end 1004 to penetrate the skin of a patient and act substantially like a needle for insertion of closure system 100 in the body of a patient.

Chamber 1000 may accommodate anchors 110 and 120 in a linear arrangement, as shown in FIG. 10. In such an arrangement, when closure system 100 is delivered into the body of a patient, anchor 110 may be delivered first, followed by anchor 120. strap 130 may be connected to anchors 110 and 120 and may form a loop within chamber 1000 behind anchors 110 and 120, as shown in FIG. 10. Upon delivery of anchors 110 and 120, the trailing portion of strap 130 may still be partially inside of chamber 1000. By withdrawing delivery system 100 from the site of delivery, the remainder of strap 130 may be removed from chamber 1000. In another embodiment, strap 130 may be held between anchors 110 and 120 within chamber 1000. In such an arrangement, anchor 110 may be delivered into the body first, followed by strap 130, then followed by anchor 120. Of course, other arrangements are within the scope of the invention.

In an embodiment, the delivery end 1004 can be designed to permit system 100 to be inserted into tube 1002. The delivery end 1004 may be sufficiently sized to permit anchors 110 and 120 to be securely positioned therewithin. Within tube 1002, the anchors 110 and 120, in one embodiment, may be placed in substantial linear alignment. In some embodiments, where anchors 110 and 120 include eyelets, the eyelets of the anchors 110 and 120 may sit substantially flush against the walls of tube 1002 to substantially fill the passageway 1008 of tube 1002. It should be appreciated that while described as a tube, the tube 1002 may be a trocar, needle, or other delivery mechanism and may have any other geometric shape as well.

Delivery system 900 may also include a stop that substantially limits how far tube 1002 can penetrate a patient's skin. For example, tube 1002 may include a protrusion 1010 about the delivery end 1004, as shown in FIG. 10. The protrusion 1010 may be designed to limit how far the tube 1002 penetrates the skin by providing a section of tube 1002 that has a relatively wide diameter. In an embodiment, the wider diameter of protrusion 1010 may act as a stopper by butting against the skin during insertion, thus preventing tube 1002 from being inserted into the patient past the point of protrusion 1010. The protrusion 1010, in an embodiment, may have any design, size, length, or thickness desired so long as the protrusion 1010 is capable of minimizing advancement of the delivery end 1004 to a desired depth so that the delivery end does not inadvertently cause damage from being inserted too far. Protrusion 1010, in one embodiment, may be fixed and/or molded directly to tube 1002. Alternatively, protrusion 1010 may be separate and/or adjustable. If adjustable, protrusion 1010 may allow for adjustment of its diameter and/or the desired depth of insertion of tube 1002. For example, the distance between delivery end 1004 and protrusion 1010 may be adjustable so as to vary the depth at which tube 1002 may be inserted into the body.

In one embodiment, the tube 1002 can be made from any material capable of passing to a site of implantation. To that end, tube 1002 may be formed from a substantially hard material so as to minimize deformation of the tube 1002 during delivery. Examples of materials that are substantially hard include metals, plastics, ceramics, or any other materials that can maintain a substantially consistent shape. In another embodiment, tube 1002 may be made of a material that is sufficiently flexible to allow for temporary deformation or curving of tube 1002 during insertion, and sufficiently hard or stiff to prevent deformation past a particular point where the curvature of tube 1002 would make deployment of closure system 100 from tube 1002 difficult. Since the tube 1002 is designed to be inserted into a human or animal body, the tube 1002, in an embodiment, can be made from a material that is biocompatible. The biocompatibility of the material may help minimize occurrence of adverse reactions due to use of the tube 1002 within the body. The tube 1002 may further include a coating on an outer surface to reduce friction between the tube 1002 and the body upon insertion. Likewise, the tube 1002 may include a coating on an inner surface to reduce friction during deployment of the system 100 situated within the tube 1002.

It should be appreciated that the tube 1002 may be provided with any shape desirable, depending on the particular application, as the shape of the tube 1002 may affect the ability of the tube 1002 to deliver the system 100 to a site of implantation. For instance, tube 1002 may be tubular in shape. In another embodiment, to the extent desired, tube 1002 may be curved to provide easier access to the site of implantation, while still allowing deployment of delivery system 100 from tube 1002. In yet another embodiment, Of course, other shapes can be used as the present invention is not intended to be limited in this manner. It should be appreciated that tube 1002 can have any shape desired as long as the tube 1002 can fit within and be advanced through body.

The tube 1002, in another embodiment, may have a length sufficient to accommodate the length of the closure system 100. In another embodiment, tube 1002 may have a length sufficient to accommodate multiple closure systems 100 and/or multiple pairs of anchors 110 and 120. It should be appreciated that tube 1002 may also have sufficient length to permit the tube 1002 to be inserted into a body and advanced through the body to a site of implantation.

The tube 1002, in one embodiment, may have a diameter sufficient to accommodate delivery of closure system 100. For example, the diameter of the tube 1002 may range from about 2 mm to about 4 mm. In one embodiment, the diameter of the tube 1002 may be 3 mm. In other embodiments, the diameter of the tube may be sufficiently larger or smaller, depending on the application, to accommodate deployment of closure system 100. The diameter may also be of a sufficient size to accommodate components and variations in design of closure system 100. For example, tube 1002 may have a diameter sufficient to accommodate anchors 110 and 120, multiple anchors, strap 130, ratchet strap 702, elongate members 802 and 804, strap 818, connectors, bushings, etc., in various arrangements and combinations. In some instances, the diameter of tube 1002 may remain substantially constant throughout. If desired, however, the diameter of the delivery mechanism 1002 may vary, as necessary. It should be appreciated that tube 1002 may have any diameter desired so long as tube 1002, together with protrusion 1010, can deliver closure system 100 to a site of implantation.

In another embodiment, looking now at FIG. 11, the delivery system 900 may include an inner chamber, such as inner tube 1102, and an outer housing, such as housing 1104. Inner tube 1102 may have a diameter sufficient large to accommodate anchors 110 and 120, and sufficiently small to allow strap 130 to be stored between inner tube 1102 and housing 1104. Accordingly, housing 1104 may have a diameter sufficient to accommodate inner tube 1102 and strap 130. As shown in FIG. 11, inner tube 1102 and housing 1104 may have lengths sufficient to accommodate multiple anchors therewithin, and to store multiple straps (e.g. straps 1112 and 1114) therebetween for delivery into the body. In FIG. 11, anchors 110 and 120, as well as other anchors, may not be visible because they may be enclosed within tube 1102.

Inner tube 1102, in an embodiment, may be tapered and/or be designed to have a sharp end to allow inner tube 1102 to act as a needle or trocar to penetrate the skin for delivery of closure system 100.

Inner tube 1102 may, in some embodiments, be attached or secured to housing 1104 so that edge 1106 of housing 1104 is recessed back from delivery end 1108 of inner tube 1102. In this way, housing 1104 may act as a stop during deployment of system 100 and/or insertion into a patient's body. For example, if delivery end 1108 of inner tube 1102 is inserted through the skin into the underlying tissue of a patient, the larger diameter of housing 1104 may prevent inner tube 1102 from being inserted past a particular depth. Inner tube 1102 may, in one embodiment, be recessed back from delivery end 1108 at a fixed distance, or may be recessed back at an adjustable or variable distance to allow for varying depths of penetration. Also, as shown, housing 1104 may have a diameter greater than that of inner tube 1102.

As noted, anchors 110 and 120 may be accommodated within inner tube 1102 for delivery into a patient. In an embodiment, it may be desirable to position strap 130 between inner tube 1102 and housing 1104 if, for example, the diameter of inner tube 1102 is sufficiently small. In this case, inner tube 1102 may include an opening, such as opening 1100 for example, that may allow strap 130 to remain attached anchor 110 and 120 when ratchet strap is stored outside of inner tube 1102 and anchors 110 and 120 are stored within. In such an embodiment, strap 130 may be connected to anchors 110 and 120, for instance, through opening 1110

In some instances, opening 1110 may be a longitudinal slot extending partly or entirely along the length of tube 1102 to allow for ejection of anchors 110 and 120 from within tube 1102, while strap 130 is stored between tube 1102 and housing 1104. For example, if anchor 110 is attached to strap 130, when anchor 110 is ejected, it may pull one end of strap 130 along opening 1100 and into the patient. Similarly, when the second anchor is delivered, it may slide the other end of strap 130 along opening 1100 and into the patient.

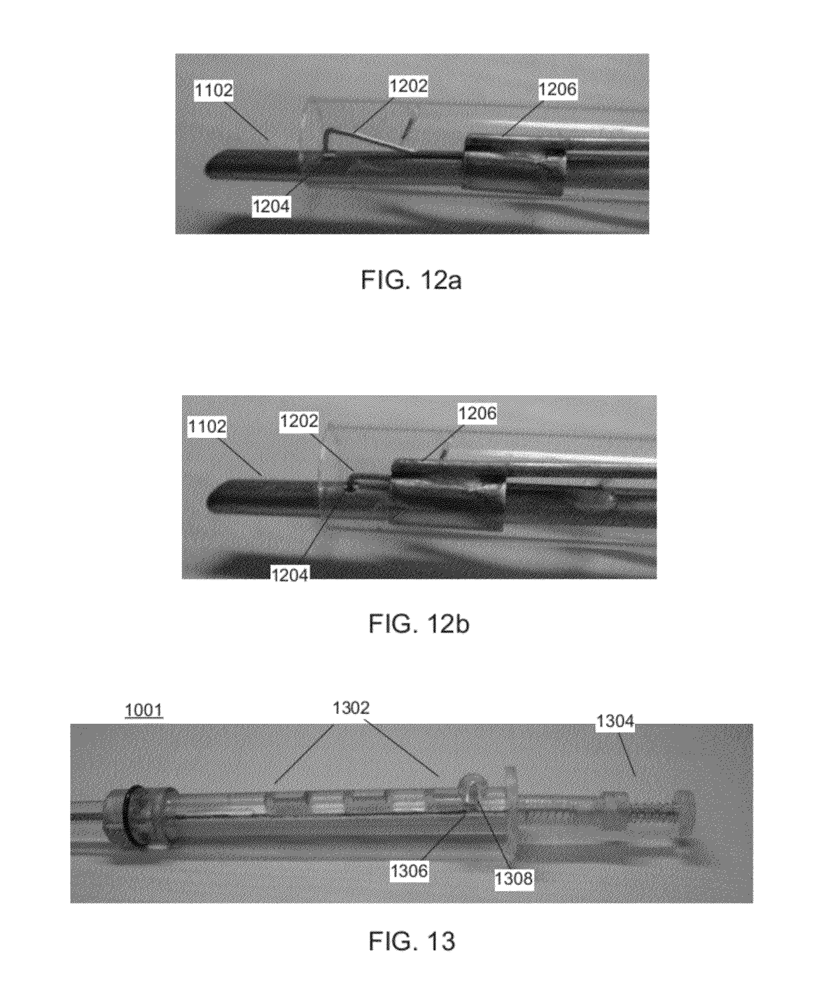

In some embodiments, delivery system 900 may include a restraining device that blocks or otherwise prevents multiple anchors from being inadvertently ejected at the same time. In one embodiment, the restraining device may include a spring loaded pin, such as pin 1202, as shown in FIGS. 12a-12b. When inserted through hole 1204, pin 1202 may block tube 1102 and prevent anchors 110 and 120 from becoming accidentally ejected from inner tube 1102. For example, if pin 1202 is blocking the pathway within inner tube 1102, anchors 110 and 120 may not be able to advance through the pathway and into the body of a patient. Alternatively, if anchor 110 has been deployed into the body, pin 1202 may be inserted through hold 1204, thus blocking second anchor 120 from being inadvertently deployed.

In an embodiment, pin 1202 may be activated (e.g. placed in a locked or open position) by a plunger (not shown) on the handle or on another remote location of delivery mechanism 1100. For example, when the plunger is positioned in a first position, it may retract translating sleeve 1206 back from the distal end of inner tube 1102, as shown in FIG. 12a, thus releasing pin 1202 and allowing it to move to an open position. Similarly, when translating sleeve is moved toward the distal end of inner tube 1102, as shown in FIG. 12b, it may push pin 1202 through hole 1204 into a locked position, thus preventing deployment of an anchor stored within inner tube 1102. Although described as a spring-loaded pin, one skilled in the art will recognize that any means of restraining anchors from becoming inadvertently deployed may be used.

Referring again to FIG. 10, in accordance with an embodiment, delivery system 900 may be provided with an ejection mechanism 1001 for deploying all or part of closure system 100 from chamber 1000 into a patient. Delivery system 900 may be able to push or otherwise eject anchors 110 and 120 and/or tension strap 130 out of tube 1002 through opening 1003 into the body of a patient undergoing surgery.

In one embodiment, ejection mechanism 1001 may include a plunger 1012 designed to apply a force to closure system 100 to direct closure system 100 from within tube 1002. In particular, the force imparted by plunger 1012 can act to push or otherwise eject closure system 100 from tube 1002, through opening 1003. In an embodiment, plunger 1012 may be coupled to a rod designed to directly push closure system 100 through tube 1002. When sufficient force is applied to plunger 1012, anchors 110 and 120 may advance along pathway 1008 of tube 1002 toward opening 1003, for delivery to the surgical site. In one embodiment, plunger 1012 may be designed to allow air pressure to enter. For example, plunger 1012 may activate a valve that allows compressed air to enter delivery mechanism 900 and push closure system 100 through opening 1003. In a like manner, plunger 1012 may also be designed to allow fluid or liquid pressure to enter. FIG. 13 shows another example of a spring loaded plunger (e.g. plunger 1304) for advancing anchors 110 and 120 along pathway 1008. Of course, other designs of the plunger 1012 may also be possible as the present invention is not intended to be limited in this manner.

In one embodiment, plunger 1012 can be situated at opposing end 1006 of tube 1002 and/or housing 1104. Of course, other locations for the plunger 1012 are possible as long as sufficient force can be applied to deploy, eject, and/or push anchors 110 and 120 from the tube 1002.

Delivery system 900 may be designed to deploy anchors 110 and 120 serially or simultaneously. As illustrated in FIG. 10, in an embodiment, to deploy anchors 110 and 120 serially, ejection mechanism 1001 may be indexed or arranged so that only one anchor may be deployed at a time. In one embodiment, ejection mechanism 100 may include a series of openings to index the plunger tube in various stages. For example, in a first stage 1016, the anchors 110 and 120 are loaded and not yet deployed. In a second stage 1018, anchor 110 may be ejected from the tube 1002. In a third stage 1020, anchor 120 may be ejected from the tube 1002. With such a design, plunger 1012 may be provided to deploy anchors 110 and 120 one at a time. To deploy anchors 110 and 120 simultaneously, plunger 1012 may include a series of compartments or slots to index the plunger tube, for instance, in only two stages, with the first stage 1016 acting to load the anchors 110 and 120 and the second stage 1018 acting to eject both anchors 110 and 120. It should be appreciated that plunger 1012 may have other designs as well.

For example, and referring now to FIG. 13, in one embodiment, ejection mechanism 1001 may include multiple indexed openings 1302 that can act to prevent plunger 1304 from directly advancing along the entire length of ejection mechanism 1001. For example, the openings may stop plunger 1304 from advancing past a point where multiple anchors are ejected.

Ejection mechanism 1001 may also include an extrusion 1306 coupled to plunger 1304. As plunger 1304 advances, extrusion 1306 may move through indexed openings 1302. As shown, indexed openings 1302 may be radially offset from one another so that, as plunger 1304 is advanced extrusion 1306 can act against the openings to halt plunger 1304 from further advancement. The indexed openings, in one embodiment, may be provided with a length that permits only one anchor to be ejected from the delivery system as plunger 1304 advances the length of the indexed opening. Once extrusion 1306 has stopped the advancement of plunger 1306, extrusion 1306 may be moved (e.g. by spherical knob 1308) to the next indexed opening, thus allowing plunger 1304 to again advance and eject another anchor. Of course, other arrangements for ensuring only one anchor is ejected at a time can be provided. For example, if plunger 1304 is actuated electronically or hydraulically, ejection mechanism 1001 may include sensors and electronics to provide plunger 1304 with only enough force to eject a single anchor.

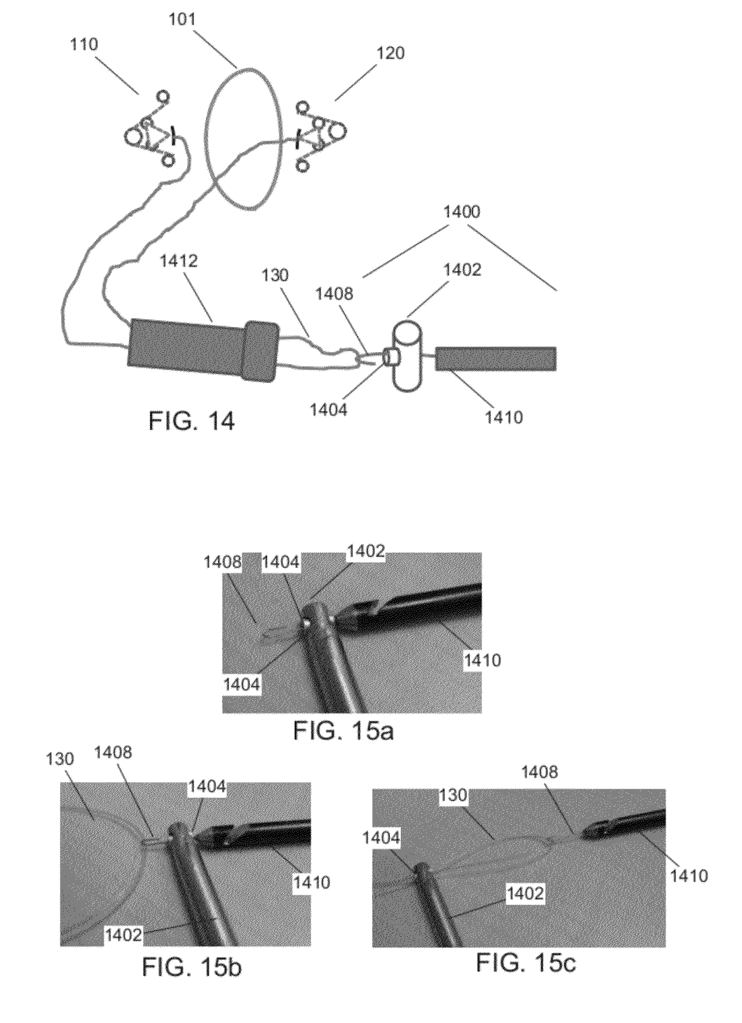

Anchors 110 and 120, as noted above, may be placed on opposing sides of the fascial defect, for example. One skilled in the art will recognize that additional pairs of anchors may also be placed so anchors in the pair are on opposing sides of the defect from each other. Tube 1002, once has been used to deliver the anchors, can be removed from the site of delivery. In one embodiment, tube 1002 may be designed so that during its removal, the suture 602 may be pulled out of the body of the patient through guide 1412, as shown in FIG. 14, allowing the suture 602 to extend outside the body of the patient.

Tensioning System

As described above, anchors 110 and 120 of closure system 100 are designed to be drawn toward each other in order to secure and close the fascial opening 101. To draw the anchors toward one another, a tensioning mechanism may be provided to exert a sufficient force on strap 130, so as to pull anchors 110 and 120 toward each other in order to substantially close the fascial defect.

For example, as shown in FIG. 14, a tensioning mechanism 1400 is illustrated that can exert a force against strap 130 to draw anchors 110 and 120 toward each other. In an embodiment, tensioning mechanism 1400 may include a rod 1402 having a hole 1404 through which strap 130 may be pulled. Pulling strap 130 through hole 1404 may effectively shorten the length of strap 130 between anchors 110 and 120, thus pulling anchors 110 and 120 closer together.

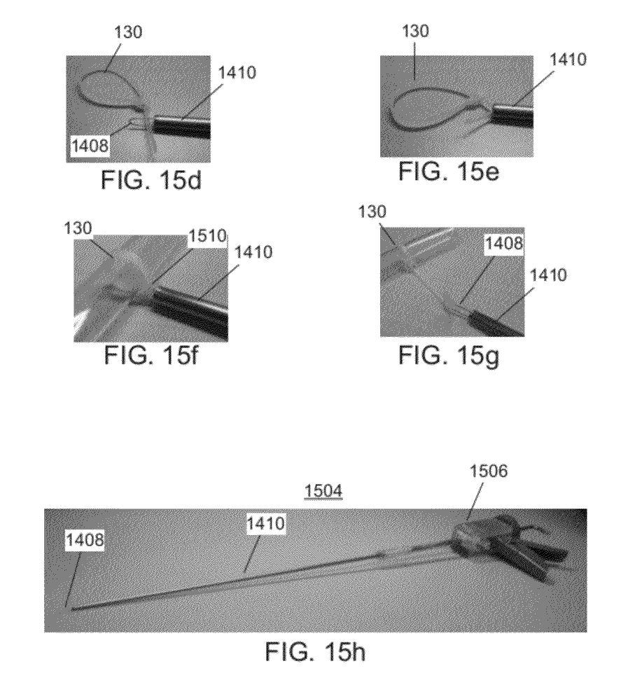

In another embodiment, as illustrated in FIGS. 15a-b, tensioning mechanism 1400 may include a bar 1410 and loop 1408 designed to pull strap 130 through rod 1402. Bushing 1403 may have an outer diameter allowing it to be inserted through and held within hole 1404. Loop 1408 may have a sufficiently small width so that it may fit through hole 1404 and bushing 1403 in order to grasp strap 130, as shown in FIG. 15a. In some instances, loop 1408 may have a hook shape to catch strap 130, as shown in FIG. 15b. Rod 1410 and loop 1408 may then be drawn back through hole 1404 in order to pull strap 130 through bushing 1403.

Bushing 1403 may be made from a material having sufficient plasticity, such as a metal, for example, so that once strap 130 is pulled through and tensioned, bushing 1403 may be crimped around strap 130 in order to secure strap 130 and maintain the tension. In some embodiments, rod 1402 may include a mechanism to crimp bushing 1403, which is discussed below.

In an embodiment, looking now at FIGS. 15c-h, securing mechanism 1400 may include a loop 1408 and bar 1410 designed to withdraw strap 130 into a hollow chamber within bar 1410. Pulling strap 130 into the hollow chamber may act to create tension on strap 130. For example, loop 1408 may extend through the hollow chamber within bar 1410 so that, when withdrawn into the chamber, loop 1408 may pull strap 130 into the chamber. FIGS. 15d-15g show an example of bar 1410 and loop 1408 designed to tighten strap 130 by drawing it into bar 1410. In FIG. 15d, loop 1408 may extend from bar 1410 so that it may be placed around strap 130. Loop 1408 may be withdrawn back through bar 1410, as shown in FIG. 15e-15f. As loop 1408 is withdrawn, it may continue to pull strap 130 to create tension.

In some instances, lock 1510 may have a sufficient size, larger than the diameter of bar 1410, for example, so that lock 1510 cannot be drawn into bar 1410. As loop 1408 is further withdrawn, bar 1410 may abut and push against lock 1510 in order to tighten and provide tension on strap 130, as shown in FIG. 15f. In FIG. 15g, wire loop may again be advanced to release strap 130 once strap 130 has sufficient tension. FIG. 15h shows an example of tensioning device 1504, having bar 1410 and loop 1408, which may be attached to and operated by handle 1506.

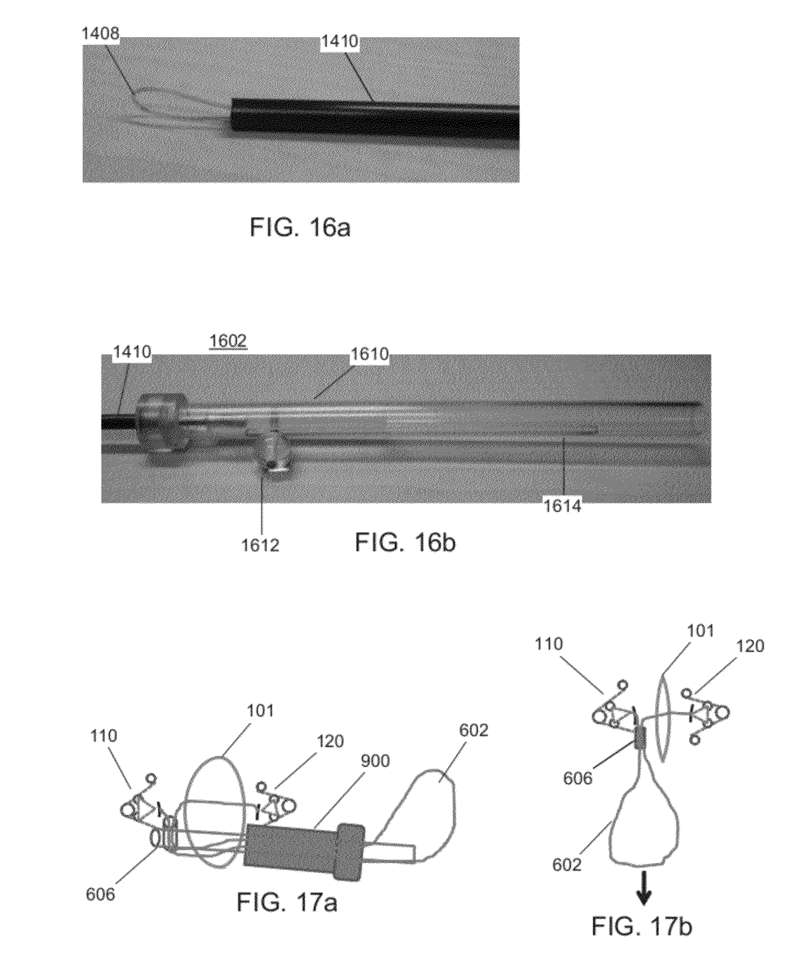

Securing mechanism 1400 may also include a slide 1612, as shown in FIGS. 16a and 16b, designed to withdraw loop 1408 through rod 1410 and provide a pulling force on strap 130. In one embodiment, rod 1410 may be coupled to tube 1610, as shown in FIG. 16b. Loop 1408 may extend through the length of rod 1410 and into tube 1610 where it may be coupled to slide 1612. In an embodiment, slide 1612 may extend through opening 1614 along the length of tube 1610, thus allowing slide 1612 to move back and forth within opening 1614 along the length of tube 1610. In such a design, as slide 1612 is drawn back, it may pull loop 1408 back through rod 1410 to provide a pulling force that may be used to tighten strap 130.

Due to its position between the anchors, when a force is exerted on tension strap 130, anchors 110 and 120 may be pulled toward each other. In one embodiment, delivery system 900 may provide a way to secure tension strap 130 so that the tension is not lost and tension strap 130 does not become slack. In particular, as described above, a ratcheting strap/device may be used to ensure tension is not lost. In another embodiment, and if tension strap 130 includes a suture, as shown in FIGS. 17a-17b, securing mechanism 606 may be designed to be advanced through the guide 940 and along the suture 602 to a site adjacent the anchors 110 and 120.

In some instances, securing mechanism 606 may be a sleeve or bushing made from a material having sufficient plasticity so that it may be crimped around strap 130. Once securing mechanism 606 is advanced to a site adjacent to one or more of the anchors placed around opening 101, as shown in FIGS. 17a-17b, securing mechanism 606 may be crimped in place in order to maintain tension on strap 130 between the anchors.

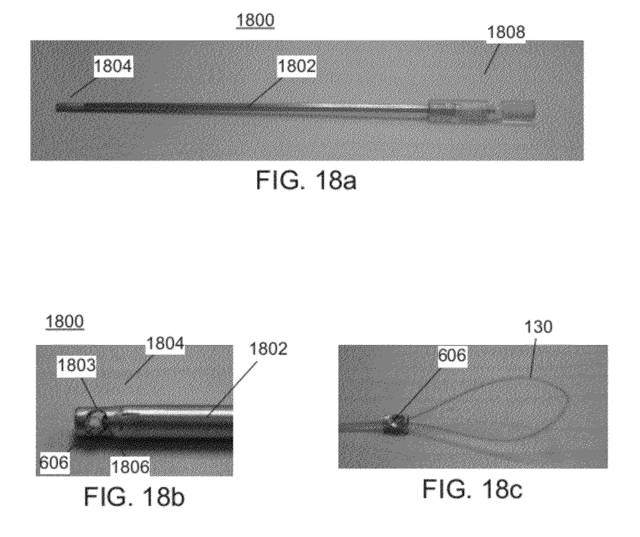

As shown in FIGS. 18a-18b, tensioning mechanism 1400 may include crimping tool 1800 to crimp securing mechanism 606 in place. Crimping tool 1800 may have an outer tube 1802 having a hole 1803 at its distal end 1804. The hole 1803 may have a diameter sufficient to accommodate securing mechanism 606, as shown in FIG. 18b. Crimping tool 1800 may also have an inner rod 1806 within outer tube 1802 and extending through the length of outer tube 1802. Inner rod 1806 may slide back and forth within outer tube 1802 in order to push against securing mechanism 606. Inner rod 1806 may also be coupled to actuation handle 1808 so that, when force is exerted on actuation handle 1808, inner rod 1806 slides within outer tube 1802 and presses against securing mechanism 606. When enough force is exerted on securing mechanism 606 by inner rod 1806, securing mechanism may deform and crimp around strap 130, as shown in FIG. 18c.

In an embodiment, actuation handle may be directly coupled to inner rod 1806 so that force applied to actuation handle 1808 is directly translated to inner rod 1806 and securing mechanism 606. In other embodiments, actuation handle 1808 may apply a force indirectly to inner rod 1806 and/or securing mechanism 606. For example, actuation handle, when engaged, by activate a fluid or gas hydraulic system that applies a crimping force to securing mechanism 606.

In an alternative embodiment, outer tube 1802 and inner rod 1806 may be threaded (not shown) so that inner rod 1806 may act as a screw. In such a design, rotation of activation handle 1808 may be translated to linear motion of inner rod 1806 through outer tube 1802. As inner rod 1806 is rotated and advanced, inner rod 1806 may exert a crimping force against securing mechanism 606. The mechanical advantage produced by the screwing action may assist in applying enough force to crimp securing mechanism 606 around strap 130

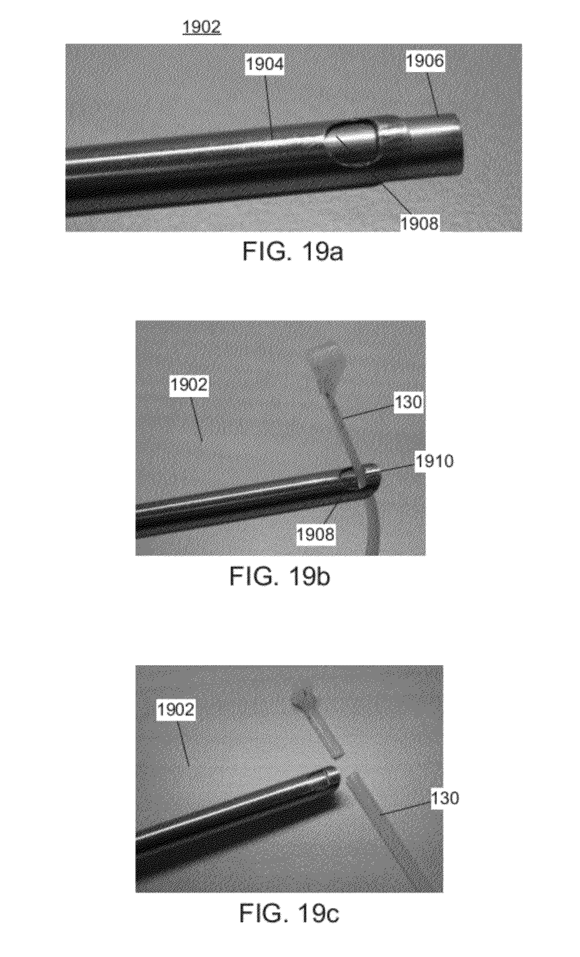

Following tensioning of strap 130, it may be desirable to sever the excess length of the strap. To do so, a cutting mechanism may be used, such as cutting mechanism 1902. As shown in FIGS. 19a-19c, the cutting mechanism 1902 may include an outer tube 1904 and an inner tube 1906 situated within the outer tube 1904. In an embodiment, the inner tube 1906 may have a diameter that allows the inner tube 1906 to fit within the outer tube 1904 and that allows the inner tube 1906 to lie against the inner walls of the outer tube 1904 and move back and forth within outer tube 1904. The cutting mechanism 1902 may be made from any substantially hard material that minimizes deformation of the cutting mechanism 1902 during severing of the strap 130.

In accordance with an embodiment, the outer tube 1904 may include an opening 1908, transverse to its surface, through which the strap 130 can be placed. The opening 1908 may be situated at one end of the outer tube 1904 although any location may be possible. The opening 1908, in an embodiment, may have any size or geometric shape desired. The opening 1908 may further be provided with a cutting edge 1910. The cutting edge 1910 may be designed so that when the when substantial force is applied to the edge 1910, the edge 1910 may sever the strap 130. The cutting edge 1910 may be a blade, an electrical wire, or any other substantially sharp edge that allows the strap 130 to be severed. It should be appreciated, that although described herein as being situated on the outer tube 1904, the opening 1908 may also be situated on the inner tube 1906.

To sever the strap 130, the outer tube 1904 and the inner tube 1906 may first be displaced from one another to allow the strap 130 to be placed through the opening 1908, as shown in FIG. 19b. Once the strap 130 is situated within the opening 1908, the outer tube 1904 and the inner tube 1906 may be moved toward one another to align themselves. As the tubes 1904, 1906 move toward one another, the inner tube 1906 may apply force to the strap 130 and push it against the cutting edge 1910. When sufficient force is applied to the strap 130 by the inner tube 1906, the strap 130 may be severed by the cutting edge 1910, as shown in FIG. 19c.

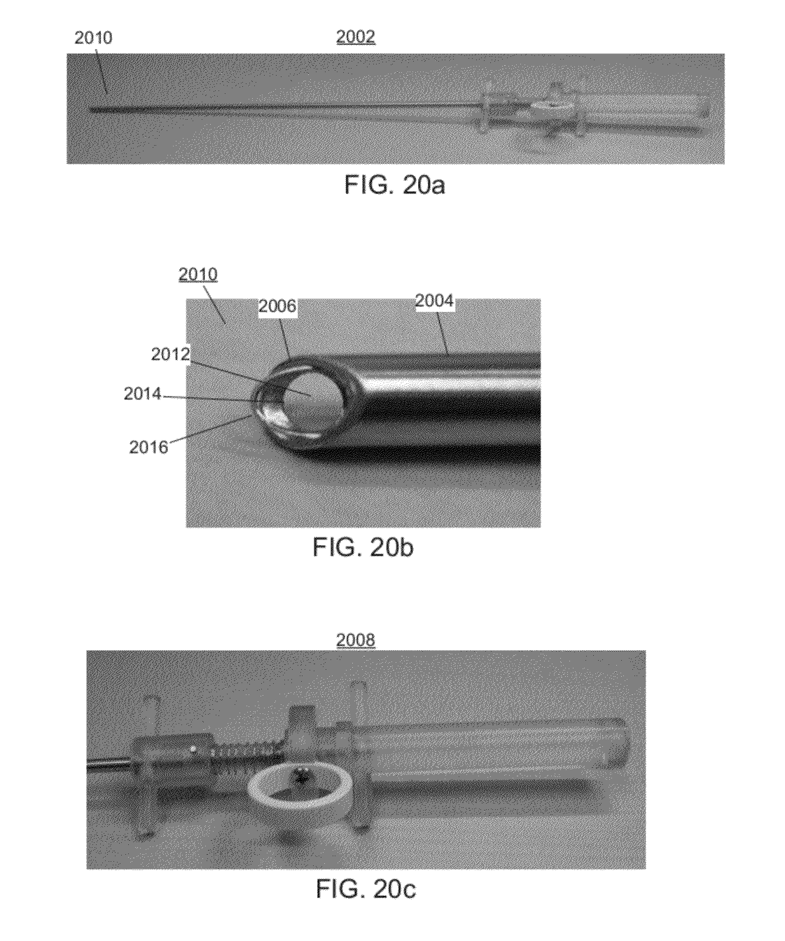

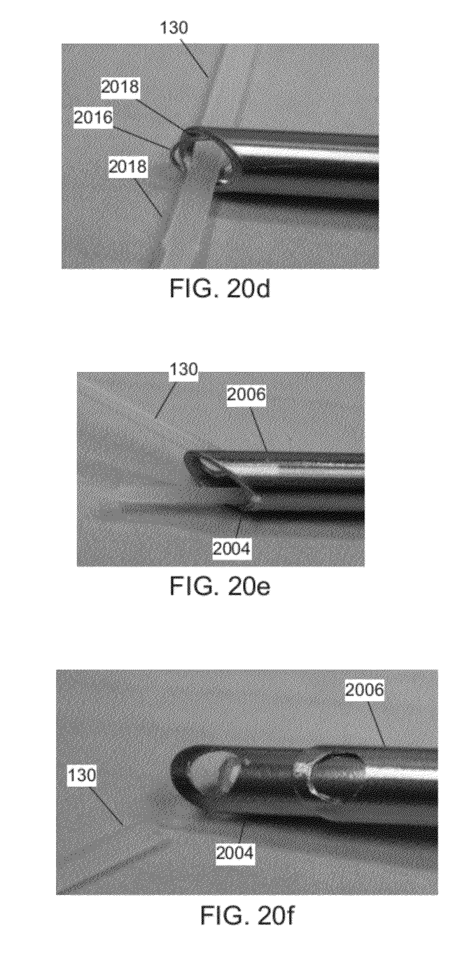

While described above as requiring separate mechanisms to tighten and then cut the strap 130, it should be appreciated that the tensioning device (e.g. tensioning device 1504 shown in FIGS. 15a-h) and the cutting mechanism 1902 may be provided in one tightening and cutting mechanism 2002, shown in FIG. 20a. The tightening and cutting mechanism 2002, in one embodiment, may include an outer tube 2004 and an inner tube 2006, which may be coupled to an actuating mechanism 2008, as shown in FIGS. 20b-20c. At its distal end 2010, the outer tube 2004 may include an opening 2012, a cutting edge 2014, as shown in FIG. 20b. Mechanism 2002 may also include hook 2016 extending from inner tube 2004 and in line with the opening 2012. The mechanism 2002 may be designed to allow the free end 2018 of strap 130 to be inserted through the opening 2012 and the hook 2016, as shown in FIG. 20d. Pulling back on the hook 2016 may bend the strap 130, causing it to fold into the tube 2004 as the strap 130 is tightened. Once the strap 130 is tightened to its desired level, the outer tube 2004 may be pulled back relative to the inner tube 2006, to shear the excess strap 130, as shown in FIGS. 20d-20f. The mechanism 2002 may then be removed from the body, and the hook 2016 may be advanced forward to release the severed portion of the strap 130.

Examples of Operation

In operation, embodiments of the disclosed systems and devices may be utilized to close a fascial defect, hernia, etc., in a patient. For example, closure system 100 may be delivered into the body of a patient by delivery system 900, through one or more laparoscopic incisions. Once delivered, tensioning mechanism 1400 may be used to close the fascial defect and maintain tension on closure system 100, so that the fascial defect does not revert to an open position.

Closure system 100 may be placed near a fascial defect in order to close the defect. For example, anchor 110 may be placed on one side of the fascial defect. Anchor 110 may initially be in a collapsed state so that it can more easily pass through a small incision in the skin. However, once through the incision, anchor may expand so that it becomes secured in its position to minimize being pulled out or being moved away from the fascial defect. Anchor 110, in one embodiment, may be placed so that its apex points away from the fascial defect. In this way, once expanded, anchor 110 will supply a greater surface area near the fascial defect, which may facilitate exerting a force on the defect in order to pull it closed. Similarly, anchor 120 may be placed on an opposing side of the fascial defect and expanded so that it becomes secured in its position. Additional anchors, or pairs of anchors, may be placed near the fascial defect in a similar manner.

A strap 130 may extend from the first anchor 110 to the second anchor 120 in the closure system 100. To that end, when a force, such as a tensioning force, is applied to the strap 130, strap 130 may act to pull the anchors toward one another. If the anchors are secured in their place on opposing sides of the fascial defect, the fascial defect may close as the anchors are pulled together. Once the defect is closed, the strap may be secured or locked in place so that it holds the fascial defect in a closed position. Although one strap 130 is disclosed, multiple straps may be used to close the defect. By holding the fascial defect closed, closure system 100 may allow a surgeon to perform other procedures to repair the defect, and may assist in the healing of the defect.

Over a period of time, once the closure system 100 is no longer needed to hold the fascial defect closed, closure system 100 may be removed from the patient. In some cases, if all or part of closure system 100 is composed of bioresorbable material, all or part of closure system 100 may be absorbed by the body.

Delivery system 900 may allow a surgeon to place closure system 100 in the body of a patient through laparoscopic incisions. For example, delivery system 900 may store closure system 100 within a chamber, such as the inner chamber of a trocar. The surgeon may direct the delivery system through a laparoscopic incision in the patients skin to the site of a fascial defect. When delivery system 100 is at a desirable location, the surgeon may use delivery system 100 to deliver a first anchor 110 on one side of the fascial defect. Once delivered, anchor 110 may expand and secure its position. The surgeon may then advance delivery system through the laparoscopic incision to an opposing side of the fascial defect. Once at a desirable position, the surgeon may use delivery system 100 to deliver the second anchor 110 to the opposing side of the fascial defect.

Once closure system 100 is delivered to the site of a fascial defect, tensioning mechanism 1400 may be used to create tension on strap 130, thus pulling anchors 110 and 120 toward each other to close the fascial defect. For example, once the anchors are in position, a surgeon may use loop 1408, for example, to attach to and grab hold of strap 130. Thereafter, upon withdrawal of loop 1480, a pulling force may be applied to strap 130 to draw anchors 110 and 120 toward each other and close the fascial defect. In some instances, strap 130 may be pulled partially or completely through a securing mechanism 606. Once enough tension has been applied to pull anchors 110 and 120 together and close the defect, securing mechanism 606 may be crimped into place in order to secure and maintain the tension on strap 130. Securing mechanism 606 may be crimped, for example, by means of crimping tool 1800, which may apply a force to securing mechanism 606 sufficient to deform and crimp securing mechanism 606 around strap 130.

In another example, if strap 130 includes bumps or teeth for ratcheting, loop 1408 may be used to pull the free end of strap 130 through a ratchet lock. In this case, strap 130 may be coupled to anchor 110, and may pass through a ratchet lock coupled to anchor 120. Securing mechanism 1400 may be used to pull strap 130 through the ratchet lock. As strap 130 is pulled, anchor 110 and 120 are pulled toward each other. The ratchet lock may prevent strap 130 from becoming slack, thus maintaining tension on strap 130 and holding the fascial defect in a closed position. Once strap 130 is under sufficient tension, a cutting tool may be used to cut excess length from strap 130 and remove the excess length from the body.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the invention.

What is claimed is:

1. A system for closing a fascial opening, the system comprising:
a first anchor and a second anchor for placement on opposing sides of a fascial opening;
a strap having one end coupled to the first anchor and an opposing end coupled to the second anchor, such that in a presence of a tensioning force being applied to the strap, the strap can act to pull the anchors toward one another to close the fascial opening;
a locking mechanism though which the strap can be complementarily received to maintain the tensioning force acting on the opposing anchors, so as to keep the fascial defect from subsequently opening.

2. A system as set forth in claim 1, wherein each anchor includes two opposing legs terminating in an apex to permit the opposing legs to be biased toward or away from one another.

3. A system as set forth in claim 2, wherein, when the legs are biased toward one another, the anchor having a sufficiently thin profile to allow the anchor to be delivered through an incision.

4. A system as set forth in claim 2, wherein, when the legs are biased away from one another, the anchor having a profile relatively wider than that of an incision through which the anchor has been directed, to allow the anchor to remain in place without being pulled through the incision.

5. A system as set forth in claim 2, wherein, the legs can be biased away from one another to form an obtuse angle.

6. A system as set forth in claim 2, wherein the legs can be biased away from one another to form a 180 degree angle to allow the anchor be positioned substantially flat against a site of implantation.

7. A system as set forth in claim 1, wherein each anchor includes one or more eyelets to which the strap can be attached.

8. A system as set forth in claim 1, wherein the first anchor includes an elongated member, extending from an apex, integral with the strap.

9. A system as set forth in claim 8, wherein the second anchor includes an elongated member coupled to the locking mechanism.

10. A system as set forth in claim 9, wherein the elongated member coupled to the second anchor includes a pivot to enable the anchor to be arranged in a first direction for insertion through the incision and a second direction for placement near the fascial defect.

11. A system as set forth in claim 1, wherein the strap has a substantially rough surface to which the locking mechanism can engage, so as to minimize movement of the strap, and thus minimizing movement of the anchors away from one another.

12. A system as set forth in claim 11, wherein the rough surface includes one of a plurality of teeth, beads, or a combination thereof against which the locking mechanism can engage.

13. A system as set forth in claim 1, wherein the locking mechanism includes a sleeve having sufficient plasticity to allow its crimping about the strap to maintain the tensioning force.

14. A system as set forth in claim 1, wherein the first and second anchors, the strap, the locking mechanism, or any combination thereof is made from a material sufficiently rigid for delivery near the fascial defect, and sufficiently pliable to be manipulated into position.

15. A system as set forth in claim 14, wherein the material includes one of metal, plastic, a bioresorbable material, or a combination thereof.

16. A method for closing a fascial opening, the method comprising:
delivering, to a one side of a fascial opening, a first anchor, and to an opposing side of the fascial opening, a second anchor connected to the first expandable anchor by a strap;
applying a tensioning force to the strap in order to pull the first and second anchors toward one another, so as to close the fascial opening;
moving a locking mechanism along the strap into proximity with the fascial opening to maintain the tension on the strap and close the fascial opening;
securing the locking mechanism in its position on the strap so as to allow the fascial opening to remain closed.

17. A method as set forth in claim 16, wherein, in the step of moving, the locking mechanism includes a ratchet lock.

18. A method as set forth in claim 16, wherein, in the step of moving, the locking mechanism includes a sleeve that can be crimped about the strap in proximity to the fascial opening.

19. A method as set forth in claim 16, wherein, in the step of moving, the locking mechanism includes cinching device that can be positioned on the strap in proximity to the fascial opening.

20. An apparatus for delivering a closure system to a fascial opening, the apparatus comprising:
a tube having a chamber within which a first anchor, second anchor, and a strap connecting the first anchor to the second anchor can be accommodated, the chamber being sufficiently sized to permit the first and second anchors to be positioned in proximity to a distal end of the tube;
an activation mechanism coupled to a proximal end of the tube, and which, when activated, directs the first anchor to one side of a fascial opening, and the second anchor to an opposing side of the fascial opening through in a distal end of the tube; and
a stopping mechanism extending into the chamber of the tube to retain the second anchor within the chamber while the first anchor is advanced through the distal end of the tube.

21. An apparatus as set forth in claim 20, wherein the tube includes a protrusion arranged circumferentially about its outer surface to limit insertion of the tube through an incision to a predetermined depth.

22. An apparatus as set forth in claim 20, wherein the opening at the distal end includes a sharpened and/or tapered edge for creating an incision.

23. An apparatus as set forth in claim 20, wherein the activation mechanism imparts a fluid force within the chamber to advance the first and second anchors through the distal end of the tube.

24. An apparatus as set forth in claim 20, wherein the activation mechanism includes a rod extending from the activation mechanism along a length of the tube, so as to push at least one of the first and second anchors through the distal end of the tube.

25. An apparatus as set forth in claim 24, wherein the stopping mechanism includes a protrusion extending from the rod through a track in the tube, such that the protrusion can act to prevent the rod from pushing both the first and second anchors through the opening at the same time.

26. An apparatus as set forth in claim 20, wherein the stopping mechanism includes a pin extending through an aperture near the distal end of the chamber to block movement of the second anchor through the opening within the chamber.

27. A method for delivering a closure system to a fascial opening, the method comprising:
providing, within a tube, a first anchor, a second anchor, and a strap connecting the first and second anchor;
deploying, from within the tube, the first anchor to a side of the fascial opening and the second anchor to an opposing side of the fascial opening; and
allowing the strap to be positioned so that a tensioning force can be applied to the strap to pull the first and second anchors toward one another to close the fascial opening.

28. A method as set forth in claim 27, wherein the step of providing includes positioning the first and second anchor within the tube to permit sequential deployment.

29. A method as set forth in claim 27, wherein the step of deploying includes imparting, on each side of the opening, an incision adjacent to a site of implantation so as to permit the anchor to be deployed through each incision.

30. A system for closing a fascial opening, the system comprising:
an elongated member having sufficiently rigid hook at one end so that, in the presence of a strap connecting a first anchor and a second anchor positioned on opposing sides of a fascial opening, the hook can exert a tensioning force on the strap so as to pull the anchors toward one another to close the fascial opening;
a handle coupled to an opposing end of the elongated member; and
a member having an aperture through which the hook can extend, the member being designed to redirect a pulling angle of the strap as the strap is pulled through the aperture by the hook, so as to pull the anchors toward each other.

31. A system as set forth in claim 30, wherein the member having the aperture is an independent member that can be positioned between the strap and the hook.

32. A system as set forth in claim 30, wherein the member having the aperture is a tubular member with the aperture being situated at its distal end, within which the elongated member having a hook can be situated.

33. A system as set forth in claim 32, wherein the hook is designed to extend through the aperture at the distal end of the tubular member, so as to allow the hook to draw the strap through the aperture and into the tubular member.

34. A system as set forth in claim 32, further including a cutting blade is an opening in the surface of the member having the aperture.

35. A system as set forth in claim 34, wherein the cutting blade is a transverse opening in an outer surface of a hollow tube.

36. A system as set forth in claim 30, further comprising a device for securing the locking mechanism about the strap in order to maintain the tension force on the strap.

37. A method for closing a fascial opening, the method comprising:
positioning, adjacent to a strap connected to a first and second anchor situated on opposing sides of a fascial opening, a member having an aperture;
extending a hook through the aperture to grasp the strap; and
pulling the strap through the aperture using the hook, while allowing the aperture to redirect a pulling angle imparted on the strap in order to draw the anchors together and close the fascial opening.

38. A method as set forth in claim 37, further comprising cutting an excess length from the strap.

39. A method as set forth in claim 37, further comprising maintaining tension on the strap to allow the anchors to remain drawn together.

40. A method as set forth in claim 39, wherein the step of maintaining includes crimping a sleeve about the strap near the location of the aperture.

41. A method as set forth in claim 39, wherein the step of maintaining includes adjusting a cinching mechanism to a position on the strap adjacent to the location of the aperture.

42. A method as set forth in claim 39, wherein the step of maintaining includes drawing the strap through a ratchet lock.

43. A system for endoscopic fascial closure comprising:
a first opposing expandable anchor and a second opposing expandable anchor, the anchors being capable of being delivered through skin at opposing sides of an opening, and capable of expanding to a predetermined size to secure the anchor at the site of delivery;
a strap extending from the first anchor to the second anchor which, upon deployment, is designed to pull the anchors toward one another so as to substantially close the opening; and
a securing mechanism to secure the opposing sides of an opening in a substantially closed position.
